(19) United States
(12) Patent Application Publication
(10) Pub. No.: US 2024/0349994 A1
(43) Pub. Date: Oct. 24, 2024

(54) SIDE-BY-SIDE ENDOSCOPE AND CAMERA CHANNEL WITH STEERING

(71) Applicant: COOK MEDICAL TECHNOLOGIES LLC, Bloomington, IN (US)

(72) Inventors: Vincent McHugo, Birdhill (IE); Liam Breen, Ballina (IE); Jonathan K. Lupton, Lupton, NC (US)

(73) Assignee: COOK MEDICAL TECHNOLOGIES LLC, BLOOMINGTON, IN (US)

(21) Appl. No.: 18/631,892

(22) Filed: Apr. 10, 2024

Related U.S. Application Data
(60) Provisional application No. 63/496,740, filed on Apr. 18, 2023.

(51) Int. Cl. A61B 1/018; A61B 1/00; A61B 1/005; A61B 1/05

(52) U.S. Cl. CPC A61B 1/018; A61B 1/00098; A61B 1/0052; A61B 1/05; A61B 1/00082; A61B 1/00085; A61B 1/00087; A61B 1/00091; A61B 1/00094

(57) ABSTRACT

A scope system includes an elongate tube defining a lumen and having a distal portion. The scope system includes at least one accessory channel including at least one tubular structure defining an accessory lumen, the at least one accessory channel movably disposed within the lumen of the elongate tube. The scope system also includes a first steerable endoscopic instrument extending through the at least one accessory lumen and including a first distal instrument end that is movable relative to a distal channel end of the at least one accessory channel. The scope system also includes a second steerable endoscopic instrument extending through the at least one accessory lumen and including a second distal instrument end that is movable relative to the distal channel end of the at least one accessory channel.

### RELATED APPLICATION

This application claims the benefit of U.S. Provisional Patent No. 63/496,740, filed Apr. 18, 2023, pending, the entirety of which is herein incorporated by reference.

### FIELD

The present disclosure relates to medical devices, and more specifically to endoscope systems.

### BACKGROUND

The statements in this section merely provide background information related to the present disclosure and may not constitute prior art.

Internal body cavities and body lumens may become blocked, or the walls surrounding them may develop growths. In some cases, removal of these blockages or growths, or other treatment thereof, may be necessary. Endoscopy or other minimally invasive techniques may be used to treat these situations.

One type of treatment includes the use of catheters or other endoscopic devices that are inserted into the body lumen or cavity and toward the area where treatment is desired. Insertion of the endoscope to the target area can allow for visualization of the target area and a determination of the desired procedure and the specific location of the area to be treated.

Endoscopes generally include a camera and a set of wheels that an operator, such as a physician, operates with a first hand (in some cases, the left hand) to control scope deflection, while the second (generally, right) hand switches between the insertion tube of the endoscope and the accessory channel in order to control scope and device advancement, respectively, through the anatomy of a patient.

The duodenoscope is a medical device used in a variety of endoscopic procedures, including endoscopic retrograde cholangio-pancreatography (ERCP). In an ERCP, a physician inserts the duodenoscope into a patient's mouth, through the patient's gastrointestinal (GI) tract, and into the duodenum until the distal end of the duodenoscope is positioned near the papilla of Vater, a small mound-like structure that acts as the entrance from the common bile duct and pancreatic duct into the duodenum. The physician then uses a variety of tools and accessories that are passed through a lumen in the duodenoscope to access the common bile duct or pancreatic duct through the papilla of Vater. A cholangioscope is a specialized endoscope that is used to visually evaluate and simultaneously therapeutically intervene in the bile duct. Available cholangioscopes may be separate, reusable, or disposable, may externally mount to a side of a duodenoscope, and may pass through an accessory channel of a duodenoscope.

Available endoscopes may include both a camera channel and a working channel. Sizes of working channels may allow for standard-diameter endoscopic instruments to be used with the endoscope. Further, the camera channel may function as both a duodenoscope and a cholangioscope. The camera channel and working channel may provide for use of the camera channel and an endoscopic instrument “side-by-side” up in the biliary tree for direct cholangioscopic visualization of the endoscopic instrument. While instruments with smaller diameters for use in working channels with smaller diameters may be available, standard-diameter instruments may often more efficiently perform a procedure, such as by taking a larger biopsy sample, disintegrating a harder stone more quickly, or removing a large stone.

However, control over the location and targeting of endoscopic instruments that are positioned side-by-side with a cholangioscope may only be possible in the forward and reverse directions, because the endoscopic instruments are side-by-side with the cholangioscope rather than within the steerable working channel of the cholangioscope. Therefore, it is desirable to have a standard-sized instrument that can be steered when the instrument is side-by-side with the cholangioscope in the biliary tree, under direction visualization of the cholangioscope.

### SUMMARY

In an example, the present disclosure provides a scope system. The scope system includes an elongate tube defining a lumen extending longitudinally therethrough, the elongate tube including a distal portion. The scope system further includes at least one accessory channel including at least one tubular structure defining an accessory lumen extending longitudinally therethrough, the at least one accessory channel movably disposed at least partially within the lumen of the elongate tube. The scope system further includes a first steerable endoscopic instrument extending at least partially through the at least one accessory channel, the first steerable endoscopic instrument including a first distal instrument end that is movable relative to a distal channel end of the at least one accessory channel. The scope system further includes a second steerable endoscopic instrument extending at least partially through the at least one accessory channel, the second steerable endoscopic instrument including a second distal instrument end that is movable relative to the distal channel end. The first steerable endoscopic instrument may include a cholangioscope. The first distal instrument end may be configured to deflect from a longitudinal axis of the elongate tube in at least two directions, wherein a deflection in each of the at least two directions is less than an angle of 180 degrees relative to the longitudinal axis. The second steerable endoscopic instrument may be selected from the group consisting of a suction device, an irrigation device, an insufflation device, a camera lens washing device, an electrohydraulic lithotripsy probe, forceps, a basket, snares, electrosurgical devices, wires, a dilation balloon, an extraction balloon, a needle knife, hemostasis clips, and laser-based devices. The second steerable endoscopic instrument may include a steerable sheath. The second steerable endoscopic instrument may include an outer sheath including two or more steering wires. The second distal instrument end may be configured to deflect from a longitudinal axis of the elongate tube in at least two directions, wherein a deflection in each of the at least two directions is less than an angle of 180 degrees relative to the longitudinal axis. The first steerable endoscopic instrument and/or the second steerable endoscopic instrument may include a steering mechanism. The steering mechanism may be selected from the group consisting of a joystick, a rack and pinion, and a thumb wheel. The second steerable endoscopic instrument may include forceps. The second steerable endoscopic instrument may include an actuator or a lever configured to open and close a first jaw and a second jaw of the forceps.

In another example, the present disclosure provides a scope system. The scope system includes an elongate tube defining a lumen extending longitudinally therethrough, the elongate tube including a distal portion. The scope system further includes at least two accessory channels, each of the at least two accessory channels including at least one tubular structure defining an accessory lumen extending longitudinally therethrough, the at least two accessory channels movably disposed at least partially within the lumen of the elongate tube. The scope system further includes a first steerable endoscopic instrument extending at least partially through a first accessory channel of the at least two accessory channels, the first steerable endoscopic instrument including a first distal instrument end that is movable relative to a distal channel end of the at least two accessory channels. The scope system further includes a second steerable endoscopic instrument extending at least partially through a second accessory channel of the at least two accessory channels, the second steerable endoscopic instrument including a second distal instrument end that is movable relative to the distal channel end. The scope system further includes a coupling mechanism configured to couple the first distal instrument end to the second distal instrument end. The coupling mechanism may include a loop on the first distal instrument end, the loop configured to envelop and tighten around the second distal instrument, or a loop on the second distal instrument end that is configured to envelop and tighten around the first distal instrument end. The first steerable endoscopic instrument may include a cholangioscope. The second steerable endoscopic instrument may be selected from the group consisting of a suction device, an irrigation device, an insufflation device, a camera lens washing device, an electrohydraulic lithotripsy probe, forces, a basket, snares, electrosurgical devices, wires, a dilation balloon, an extraction balloon, a needle knife, hemostasis clips, and laser-based devices. The first steerable endoscopic instrument and/or the second steerable endoscopic instrument may include a steering mechanism selected from the group consisting of a joystick, a rack and pinion, and a thumb wheel. The second steerable endoscopic instrument may include forceps and a lever configured to open and close a first jaw and a second jaw of the forceps.

In yet another example, the present disclosure provides a method for performing endoscopy in a bile duct of a mammal. The method includes providing a scope system including: an elongate tube defining a lumen extending longitudinally therethrough, the elongate tube including a distal portion; at least one accessory channel including at least one tubular structure defining an accessory lumen extending longitudinally therethrough, the at least one accessory channel movably disposed at least partially within the lumen of the elongate tube; a first steerable endoscopic instrument extending at least partially through the at least one accessory channel, the first steerable endoscopic instrument including a first distal instrument end that is movable relative to a distal channel end of the at least one accessory channel; and a second steerable endoscopic instrument extending at least partially through the at least one accessory channel, the second steerable endoscopic instrument including a second distal instrument end that is movable relative to the distal channel end of the at least one accessory channel. The method further includes steering the first distal instrument end and the second distal instrument end separately within the bile duct. The second steerable endoscopic instrument may include a cholangioscope and the first steerable endoscopic instrument may be selected from the group consisting of a suction device, an irrigation device, an insufflation device, a camera lens washing device, an electrohydraulic lithotripsy probe, forceps, a basket, snares, electrosurgical devices, wires, a dilation balloon, an extraction balloon, a needle knife, hemostasis clips, and laser-based devices. The method may further include translating the second distal instrument end distally through a loop on the first distal instrument end; tightening the loop around the second distal instrument end; and steering the first distal instrument end and the second distal instrument end together within the bile duct.

Further areas of applicability will become apparent from the description provided herein. It should be understood that the description and specific examples are intended for purposes of illustration only, and are not intended to limit the scope of the present disclosure.

### BRIEF DESCRIPTION OF DRAWINGS

In order that the present disclosure may be well understood, there will now be described various forms thereof, given by way of example, reference being made to the accompanying drawings. The components in the figures are not necessarily to scale. Moreover, in the figures, like-referenced numerals designate corresponding parts through the different views.

FIG. 1 illustrates a partial detailed view of an example of a distal portion of an endoscope system in a side-facing configuration, according to the principles of the present disclosure;

FIG. 2 illustrates a partial side view of another example of a distal portion of an endoscope system in a biliary tree of a mammal, with only the pivot arm and accessory channels illustrated in a forward-facing configuration, according to the principles of the present disclosure;

FIG. 3 illustrates a longitudinal cross-sectional view of an example of a standard endoscopic instrument shaft;

FIG. 4 illustrates a longitudinal cross-sectional view of an example of a steerable endoscopic instrument shaft, according to the principles of the present disclosure;

FIG. 5 illustrates a side view of an example of a steerable endoscopic instrument including forceps, according to the principles of the present disclosure;

FIG. 6 illustrates a perspective view of another example of a joystick;

FIG. 7 illustrates a partial perspective view of another example of a steerable endoscopic instrument, according to the principles of the present disclosure;

FIG. 8 illustrates a partial perspective view of a distal end of an example of a camera catheter, according to the principles of the present disclosure;

FIG. 9 illustrates a partial side view of yet another example of a distal portion of an endoscope system, with only the pivot arm and accessory channels illustrated in a forward facing configuration, with loops on the distal section of an instrument shaft through which the distal end of a second steerable instrument may pass, according to the principles of the present disclosure;

FIG. 10 illustrates a partial side view of the example of the distal portion of an endoscope system illustrated in FIG. 9, with the loops tightened around the distal end of the another steerable instrument to provide for steering of the instrument and the second instrument together, according to the principles of the present disclosure; and

FIG. 11 illustrates an example of an endoscope system.

The drawings described herein are for illustration purposes only and are not intended to limit the scope of the present disclosure in any way.

### DETAILED DESCRIPTION

The following description presents examples and is not intended to limit the present disclosure, application, or uses. It should be understood that throughout the drawings, corresponding reference numerals indicate like or corresponding parts and features.

In adding reference denotations to elements of each drawing, although the same elements are displayed on a different drawing, it should be noted that the same elements have the same denotations. In addition, in describing one aspect of the present disclosure, if it is determined that a detailed description of related well-known configurations or functions blurs the gist of one aspect of the present disclosure, it will be omitted.

In the following discussion, the terms “proximal” and “distal” will be used to describe the opposing axial ends of the device, as well as the axial ends of various component features. The term “proximal” is used in its conventional sense to refer to the end of the device (or component) that is closest to the medical professional during use of the assembly. The term “distal” is used in its conventional sense to refer to the end of the device (or component) that is initially inserted into the patient, or that is closest to the patient during use. The term “longitudinal” will be used to refer to an axis that aligns with the proximal-distal axis of the device (or component). The terms “radially” and “radial” will be used to refer to elements, surfaces, or assemblies relative to one another that may extend perpendicularly from a longitudinal axis. The terms “circumference,” “circumferentially,” and “circumferential” will be used to refer to elements, surfaces, or assemblies relative to one another encircling a longitudinal axis at a radius.

The term “mammal” refers to a vertebrate animal of the zoological class Mammalia, characterized by the presence of milk-producing mammary glands for feeding young, a neocortex region of the brain, fur or hair, and three middle ear bones. Examples of mammals may include humans, dogs, and cats.

The use of the terms “a” and “an” and “the” and similar referents in the context of describing the present disclosure (especially in the context of the following claims) are to be construed to cover both the singular and the plural, unless otherwise indicated herein or clearly contradicted by context. The use of the term “plurality of” is defined by the Applicant in the broadest sense, superseding any other implied definitions or limitations hereinbefore or hereinafter unless expressly asserted by the Applicant to the contrary, to mean a quantity of more than one. Recitations of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

As used herein, the terms “comprise(s),” “include(s),” “having,” “has,” “can,” “contain(s),” and variants thereof, are intended to be open-ended transitional phrases, terms, or words that do not preclude the possibility of additional acts or structures. The present description also contemplates other examples “comprising,” “consisting of,” and “consisting essentially of,” the examples or elements presented herein, whether explicitly set forth or not.

In describing elements of the present disclosure, the terms 1^{st}, 2^{nd}, first, second, A, B, (a), (b), and the like may be used herein. These terms are only used to distinguish one element from another element, but do not limit the corresponding elements irrespective of the nature or order of the corresponding elements.

Unless otherwise defined, all terms used herein, including technical or scientific terms, have the same meanings as those generally understood by those skilled in the art to which the present disclosure pertains. Such terms as those defined in a generally used dictionary are to be interpreted as having meanings equal to the contextual meanings in the relevant field of art.

As used herein, the term “about,” when used in the context of a numerical value or range set forth means a variation of ±15%, or less, of the numerical value. For example, a value differing by ±15%, ±14%, ±10%, or ±5%, among others, would satisfy the definition of “about,” unless more narrowly defined in particular instances.

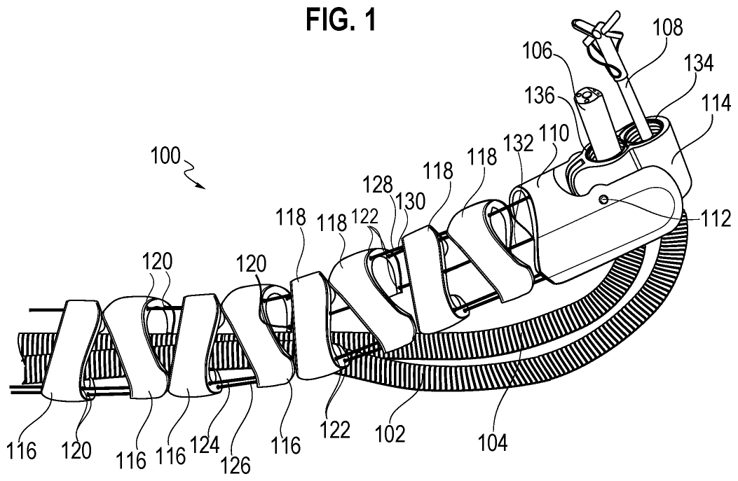

Referring to FIG. 1, a detailed view of an example of a distal portion 100 of an endoscope system 1100 in a side-facing configuration is illustrated. Distal portion 100 includes first accessory channel 102 and second accessory channel 104, each of which has a tubular structure defining an accessory lumen running therethrough. First accessory channel 102 and second accessory channel 104 may be designed as individual elongated tubes that may be movable within lumen 1108 of endoscope system 1100, thus allowing longitudinal movement of first accessory channel 102 and second accessory channel 104 with respect to central portion 1102 of endoscope system 1100. While the example illustrated in FIG. 1 includes first accessory channel 102 and second accessory channels 104, one or even three or more accessory channels may be used. For example, a single, larger accessory channel may be used to accommodate larger endoscopic tools. Further, in lieu of individual first accessory channel 102 and second accessory channel 104, a single elongate tube may be used with two or more lumens running through it. First accessory channel 102 and second accessory channel 104 may range in diameter anywhere from about 1 to about 10 millimeters.

In certain examples, first accessory channel 102 may range in diameter from about 1 millimeters to about 9.5 millimeters, or to about 9.0 millimeters, or to about 8.5 millimeters, or to about 8.0 millimeters, or to about 7.5 millimeters, or to about 7.0 millimeters, or to about 6.5 millimeters, or to about 6.0 millimeters, or to about 5.5 millimeters, or to about 5.0 millimeters, or to about 4.5 millimeters; or from about 1.5 millimeters, or from about 2.0 millimeters, or from about 2.5 millimeters, or from about 3.0 millimeters, or from about 3.5 millimeters, or from about 4.0 millimeters; or from any one of the above minima to any one of the above maxima. In a particular example, first accessory channel 102 may have a diameter of about 4.2 millimeters.

In certain examples, second accessory channel 104 may range in diameter from about 1 millimeters to about 9.5 millimeters, or to about 9.0 millimeters, or to about 8.5 millimeters, or to about 8.0 millimeters, or to about 7.5 millimeters, or to about 7.0 millimeters, or to about 6.5 millimeters, or to about 6.0 millimeters, or to about 5.5 millimeters, or to about 5.0 millimeters, or to about 4.5 millimeters, or to about 4.0 millimeters; or from about 1.5 millimeters, or from about 2.0 millimeters, or from about 2.5 millimeters, or from about 3.5 millimeters; or from any one of the above minima to any one of the above maxima. In a particular example, second accessory channel 104 may have a diameter of about 3.7 millimeters.

Distal portion 100 may have a flexible rib-like construction with a plurality individual ring-shaped ribs 116 and a plurality of circumferentially uncontinuous ribs 118 connected together to create elongate tube 1106 defining lumen 1108. Ring-shaped ribs 116 and circumferentially uncontinuous ribs 118 may be made of a variety of materials, such as polycarbonate, nylon, polyethylene, polypropylene, and polyoxymethylene. First accessory channel 102 and second accessory channel 104 may travel through a plurality ring-shaped ribs 116 and a plurality of circumferentially uncontinuous ribs 118 to distal end section 110 of distal portion 100, ring-shaped ribs 116 encircling lumen 1108 through which first accessory channel 102 and second accessory channel 104 travel. A plurality of circumferentially uncontinuous ribs 118 are distal to a plurality of ring-shaped ribs 116. Each of the plurality of circumferentially uncontinuous ribs 118 is C-shaped and includes an opening resulting in the ribs 118 being circumferentially uncontinuous. The rib openings of the plurality of circumferentially uncontinuous ribs 118 are aligned coaxially to parallel the longitudinal axis, such that first accessory channel 102 and second accessory channel 104 are reversibly movable through the coaxial rib openings into and out of the circumferentially uncontinuous ribs 118.

Distal end section 110 includes pivot pin 112 perpendicular to the longitudinal axis. Distal end section 110 includes pivot arm 114 with first accessory lumen 134 and second accessory lumen 136. Distal channel ends of first accessory channel 102 and second accessory channel 104 are disposed within respective first accessory lumen 134 and second accessory lumen 136 and secured to pivot arm 114. Instrument 108 is disposed in first accessory channel 102 and may extend distally beyond the distal end of first accessory lumen 134. Instrument 108 may translate proximally and distally relative to first accessory lumen 134 and first accessory channel 102. Camera catheter 106 is disposed in second accessory channel 104 and may extend distally beyond the distal end of second accessory lumen 136. Camera catheter 106 may translate proximally and distally relative to second accessory lumen 136 and second accessory channel 104. Pivot arm 114 is connected to distal end section 110 by pivot pin 112. Pivot pin 112 may create a pivot point, about which pivot arm 114 may rotate with respect to distal end section 110. Pivot arm 114 may be moved between a forward-viewing configuration and a side-viewing configuration as illustrated in FIG. 1.

Examples of instrument 108 may include forceps, electrosurgical devices such as sphincteromes, wires, dilation balloons, extraction balloons, needle knives, hemostasis clips, a suction device, an irrigation device, an insufflation device, a camera lens washing device, an electrohydraulic lithotripsy (“EHL”) probe, a basket, snares, laser based devices such as lithotripsy laser fibers, and any other catheter-based tool may be inserted into first accessory channel 102.

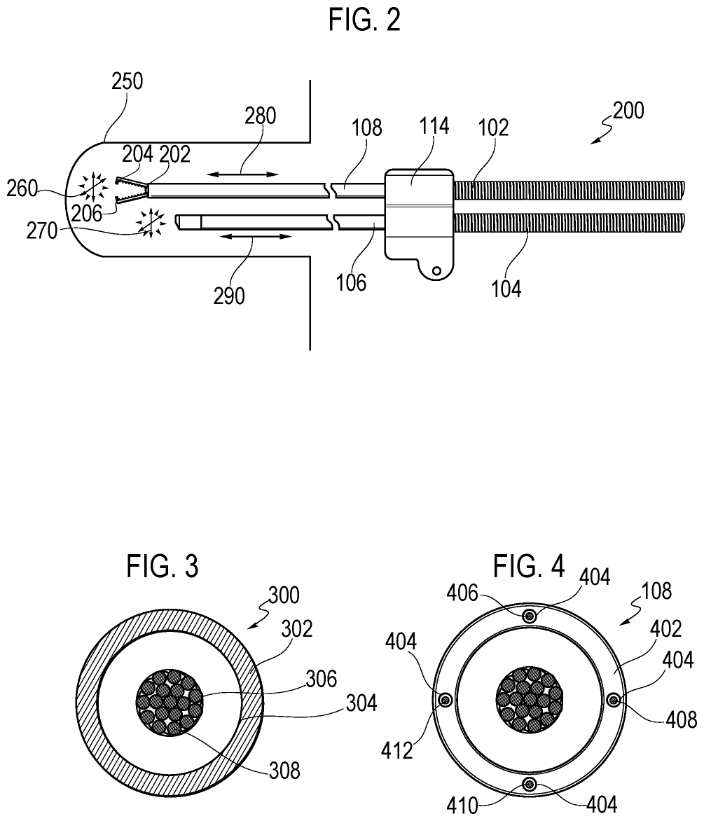

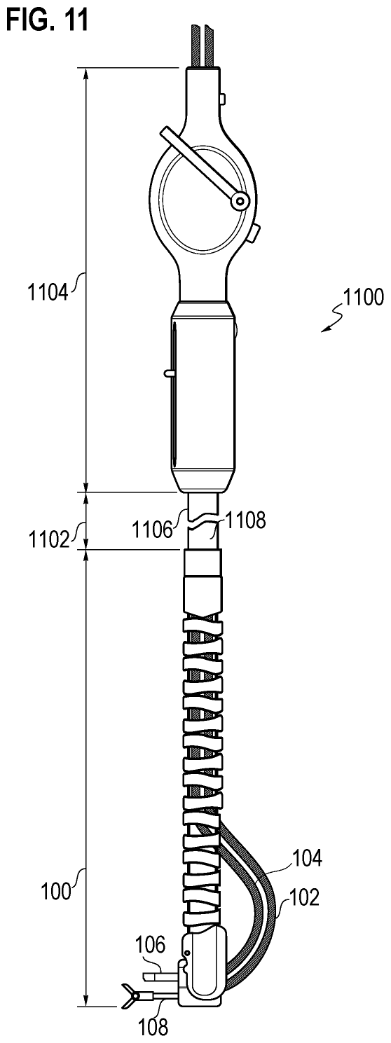

The distal ends of first accessory channel 102 and second accessory channel 104 are secured to pivot arm 114. Therefore, first accessory channel 102 and second accessory channel 104 may rotate with pivot arm 114 when moving pivot arm 114 between the side-viewing and forward-viewing configurations. FIGS. 1 and 11 illustrate first accessory channel 102 and second accessory channel 104 in the side-viewing configuration, while FIGS. 2, 9, and 10 illustrate first accessory channel 102 and second accessory channel 104 in the forward-viewing configuration.

As illustrated in FIG. 1, when in the side-viewing configuration and due to the rotation of pivot arm 114, distal portions of first accessory channel 102 and second accessory channel 104 are bent outside of the confines of plurality of circumferentially uncontinuous ribs 118 and then curve back toward and into pivot arm 114. Thus, in the forward-viewing configuration, the angle of curvature or bending radius of distal portion 100, or angle of distal portion curvature, is the same as the angle of curvature of first accessory channel 102 and second accessory channel 104 such that first accessory channel 102 and second accessory channel 104 and distal portion 100 of endoscope system 1100 are substantially parallel; but in the side-viewing configuration, the angle of curvature or bending radius of first accessory channel 102 and second accessory channel 104 is greater than the angle of curvature of distal portion 100 such that the distal portions of first accessory channel 102 and second accessory channel 104 extend outside lumen 1108 of distal portion 100.

To move pivot arm 114 from the forward-viewing configuration to the side-viewing configuration, first accessory channel 102 and second accessory channel 104 may be pushed in a distal direction relative to proximal portion 1104 and central portion 1102, which applies a force through first accessory channel 102 and second accessory channel 104 to pivot arm 114. The resulting force causes pivot arm 114 to rotate about the pivot point of pivot pin 112, thereby moving first accessory channel 102 and second accessory channel 104 and pivot arm 114 into the side-viewing configuration. To move back to the forward-viewing configuration, a proximal force may be applied to first accessory channel 102 and second accessory channel 104 relative to proximal portion 1104 and central portion 1102, thereby transferring the proximal force to pivot arm 114. The proximal force then causes pivot arm 114 to again rotate around the pivot point of pivot pin 112 in the opposite direction, thereby moving first accessory channel 102 and second accessory channel 104 and pivot arm 114 back to the forward-viewing configuration. To ensure that first accessory channel 102 and second accessory channel 104 move in unison during movements, first accessory channel 102 and second accessory channel 104 may be secured together at any point along the length of endoscope system 1100, or even along the entire length. In certain examples, first accessory channel 102 and second accessory channel 104 may be secured together using plastic tubing throughout the entire length of central portion 1102. In other examples, first accessory channel 102 and second accessory channel 104 may be secured together at the portions of first accessory channel 102 and second accessory channel 104 that extend outside the constraints of distal portion 100 when endoscope system 1100 is in the side-viewing configuration.

In addition to the ability to switch between forward-viewing and side-viewing configurations, distal portion 100 of endoscope system 1100 may also bend and rotate as desired. FIG. 1 illustrates distal portion 100 in a bent configuration, while FIG. 11 illustrates distal portion 100 in a straight configuration. The plurality of ring-shaped ribs 116 each include at least three ring-shaped rib bores 120 longitudinally traversing each of the plurality of ring-shaped ribs 116 from distal surface to proximal surface of each of the plurality of ring-shaped ribs 116. Ring-shaped rib bores 120 may be low friction bores. However, each of the plurality of ring-shaped ribs 116 may include four, five, or more ring-shaped rib bores 120 longitudinally traversing each of the plurality of ring-shaped ribs 116 from distal surface to proximal surface of each of the plurality of ring-shaped ribs 116. The plurality of circumferentially uncontinuous ribs 118 each include at least three circumferentially uncontinuous rib bores 122 longitudinally traversing each of the plurality of circumferentially uncontinuous ribs 118 from distal surface to proximal surface of each of the plurality of circumferentially uncontinuous ribs 118. Circumferentially uncontinuous rib bores 122 may be low friction bores. However, each of the plurality of circumferentially uncontinuous ribs 118 may include four, five, or more circumferentially uncontinuous bores 122 longitudinally traversing each of the plurality of circumferentially uncontinuous ribs 118.

Endoscope system 100 includes first drive member 124, second drive member 126, and third drive member 128. Each of first drive member 124, second drive member 126, and third drive member 128 extends through one of the ring-shaped rib bores 120 in each of the plurality of ring-shaped ribs 116 and through one of the circumferentially uncontinuous rib bores 122 in each of the plurality of circumferentially uncontinuous ribs 118. Each of first drive member 124, second drive member 126, and third drive member 128 may be fixedly attached to distal end section 110 and extend through one ring-shaped rib bore 120 in each of the plurality of ring-shaped ribs 116 and one circumferentially uncontinuous rib bore 122 in each of the plurality of circumferentially uncontinuous ribs 118 to handle portion 1104. Alternatively, each of first drive member 124, second drive member 126, and third drive member 128 may be fixedly attached to distal end section 110 and extend through or outside of lumen 1108 to handle portion 1104. First drive member 124 may be fixed on a wall of distal end section 110 while second drive member 126 and third drive member 128 may be fixed on opposing walls of distal end section 110 with respect to first drive member 124.

As illustrated in FIG. 1, endoscope system 100 may include fourth drive member 130 and may also include fifth drive member 132. Fourth drive member 130 be fixedly attached to distal end section 110 and extend through one ring-shaped rib bore 120 in each of the plurality of ring-shaped ribs 116 and one circumferentially uncontinuous rib bore 122 in each of the plurality of circumferentially uncontinuous ribs 118 to handle portion 1104. Alternatively, fourth drive member 130 may be fixedly attached to distal end section 110 and extend through or outside of lumen 1108 to handle portion 1104. Fifth drive member 132 be fixedly attached to distal end section 110 and extend through one ring-shaped rib bore 120 in each of the plurality of ring-shaped ribs 116 and one circumferentially uncontinuous rib bore 122 in each of the plurality of circumferentially uncontinuous ribs 118 to handle portion 1104. Alternatively, fifth drive member 132 may be fixedly attached to distal end section 110 and extend through or outside of lumen 1108 to handle portion 1104.

In an example, to move distal portion 100 from the straight configuration illustrated in FIG. 11 to the bent configuration illustrated in FIG. 1, first drive member 124 may be pulled in a proximal direction. This proximal movement of first drive member 124 may result in a force being applied through first drive member 124 and to distal end section 110. This force may cause the flexible, ribbed body of distal portion 100 to bend toward the configuration illustrated in FIG. 1. To move distal portion 100 back to the straight configuration from the bent configuration, second drive member 126 and third drive member 128 may be pulled in a proximal direction. Because second drive member 126 and third drive member 128 are connected to the opposite side of distal end section 110, a force is applied through second drive member 126 and third drive member 128 and to distal end section 110 that may move distal portion 100 back towards the straight configuration. Fourth drive member 130 and fifth drive member 132 illustrated in FIG. 1 may also operate to move distal portion 100 from the straight configuration to the bent configuration or from the bent configuration to the straight configuration.

First drive member 124, second drive member 126, and third drive member 128 may also be used to secure together each of the plurality of ring-shaped ribs 116 and to secure together each of the plurality of circumferentially uncontinuous ribs 118 and to secure together the plurality of ring-shaped ribs 116 with the plurality of circumferentially uncontinuous ribs 118. Sufficient tension may be supplied to first drive member 124, second drive member 126, and third drive member 128 thereby securing together the plurality of ring-shaped ribs 116 and the plurality of circumferentially uncontinuous ribs 118 along first drive member 124, second drive member 126, and third drive member 128. Fourth drive member 130 and fifth drive member 132 illustrated in FIG. 1 may also be used to secure together each of the plurality of ring-shaped ribs 116 and to secure together each of the plurality of circumferentially uncontinuous ribs 118 and to secure together the plurality of ring-shaped ribs 116 with the plurality of circumferentially uncontinuous ribs 118. Sufficient tension may be supplied to fourth drive member 130 and fifth drive member 132 thereby securing together the plurality of ring-shaped ribs 116 and the plurality of circumferentially uncontinuous ribs 118 along fourth drive member 130 and fifth drive member 132. Each of the plurality of ring-shaped ribs 116 and circumferentially uncontinuous ribs 118 may be shaped to allow for minimal contact between each pair of ring-shaped ribs 116, between each pair of circumferentially uncontinuous ribs 118, and between a distal-most ring-shaped rib 116 and a proximal-most circumferentially uncontinuous rib 118, thereby minimizing friction and allowing for easier bending of distal portion 100 to the bent configuration and maximum flexibility.

Second drive member 126 or third drive member 128 may also include built-in electrical wiring that allows second drive member 126 or third drive member 128 to function as a circuit for an LED light.

The plurality of ring-shaped ribs 116 and circumferentially uncontinuous ribs 118 may be covered by a protective sleeve (not shown) that may be made up of various biocompatible materials, such as an elastic material. The protective sleeve may protect the plurality of ring-shaped ribs 116 and circumferentially uncontinuous ribs 118 while also preventing body tissue from actually being pinched between ring-shaped ribs 116 or between circumferentially uncontinuous ribs 118 or between ring-shaped rib 116 and circumferentially uncontinuous rib 118 when distal portion 100 is moved between the bent configuration and the straight configuration. The protective sleep may also include a slot that corresponds to the openings in the plurality of circumferentially uncontinuous ribs 118 that allows first accessory channel 102 and second accessory channel 104 to move outside of the protective sleeve and between the forward-viewing configuration and the side-viewing configuration. The protective sleeve may also help with torque transmission when moving distal portion 100 between the bent and straight configurations. Some natural lag may occur when manipulating first drive member 124, second drive member 126, and third drive member 128 that may cause part of distal portion 100 to move first, while the rest of distal portion 100 lags behind, but eventually moves as well. The protective sleeve may ensure that entire distal portion 100 moves together and with minimal lag.

Endoscope system 100 may move between a bent configuration and a straight configuration while endoscope system 100 is also in either the forward-facing or side-facing configurations. Endoscope system 100 may be manipulated and used in any combination of the above mentioned configurations, and may be repeatedly movable between all configurations.

Referring to FIG. 2, a partial side view of another example of a distal portion 200 of an endoscope system is illustrated inserted into a biliary tree 250 of a mammal. Camera catheter 106 may be translated proximally and distally relative to pivot arm 114 according to bidirectional arrow 290, and is “steerable,” in that camera catheter 106 may be deflected in multiple directions. As exemplified in FIG. 2 by arrow 270, camera catheter 106 may be deflected in various directions relative to an axis parallel to bidirectional arrow 290, including more than two, three, four, six, eight, twelve, twenty-four, or thirty-six directions, and up to 360 directions relative to an axis parallel to bidirectional arrow 290. The degree of deflection relative to the axis parallel to bidirectional arrow 290 may be less than 180 degrees, or less than 175 degrees, or less than 170 degrees, or less than 165 degrees, or less than 160 degrees, or less than 155 degrees, or less than 150 degrees, or less than 145 degrees, or less than 140 degrees, or less than 135 degrees, or less than 130 degrees, or less than 125 degrees, or less than 120 degrees, or less than 115 degrees, or less than 110 degrees, or less than 105 degrees, or less than 100 degrees, or less than 95 degrees, or less than 90 degrees.

Instrument 108 may be translated proximally and distally relative to pivot arm 114 according to bidirectional arrow 280. In the example of distal portion 200 illustrated in FIG. 2, instrument 108 is forceps 202, including upper jaw 204 and lower jaw 206. Instrument 108 is “steerable,” in that instrument 108 may be deflected in multiple directions. As illustrated in FIG. 2 by arrow 260, instrument 108 may be deflected in various directions relative to an axis parallel to bidirectional arrow 280, including more than two, three, four, six, eight, twelve, twenty-four, or thirty-six directions, and up to 360 directions relative to an axis parallel to bidirectional arrow 280.

Referring to FIG. 3, a longitudinal cross-sectional view of an example of a standard endoscopic instrument shaft 300. Shaft 300 includes an outer sheath 302 that is typically a coil. Within a center of inner sheath 304 is an inner drive wire 306 made up of a plurality of braided wires 308.

Referring to FIG. 4, a longitudinal cross-sectional view of an example of a shaft of an instrument 108 that is a steerable endoscopic instrument is illustrated. Outer sheath 402 includes a plurality of steering lumens 404 parallel to a longitudinal proximal-distal axis of instrument 108 and distributed evenly around the cross-sectional circumference of outer sheath 402. In an example, two steering lumens 404 would be distributed 180° apart about the cross-sectional circumference of outer sheath 402. In another example, three steering lumens 404 would be distributed 120° apart about the cross-sectional circumference of outer sheath 402. In the example illustrated in FIG. 2, four steering lumens 404 are distributed 90° apart about the cross-sectional circumference of outer sheath 402. In still other examples, five steering lumens 404, six steering lumens 404, or more steering lumens 404 may be distributed evenly apart about the cross-sectional circumference of outer sheath 402. A steering wire extends throughout the longitudinal length of each steering lumen 404. In the example illustrated in FIG. 4, each of steering wires 406, 408, 410, and 412 extends through a steering lumen 404 such that proximal force applied on steering wire 406, 408, 410, or 412 results in deflection of the shaft of instrument 108 in the direction of steering lumen 404 through which steering wire 406, 408, 410, or 412 extends and steering wires 406, 408, 410, and 412 are configured to deflect the shaft of instrument 108. It may be beneficial that instrument 108 is wire-guided to allow swift insertion and removal of instrument 108 to a target site, because the papilla of the biliary duct may not be under direct visualization to aid in cannulation of instrument 108 from an endoscope system 100 into the biliary duct when the camera catheter is up in the biliary duct.

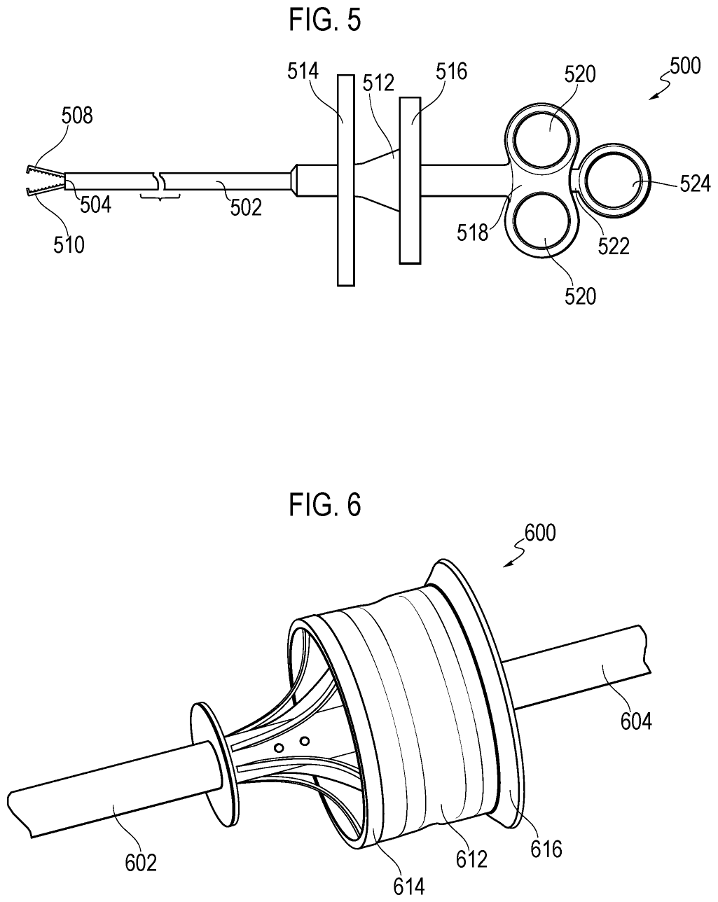

Referring to FIG. 5, a side view of an example of a steerable endoscopic instrument 500 including forceps is illustrated. Upper jaw 508 and lower jaw 510 of forceps are illustrated as open. Shaft 502 may be made of a flexible material and have a distal end 504 connected to a steering mechanism. Examples of steering mechanisms may include joystick 512 as illustrated in FIG. 5, a rack that may operate with a pinion, and one or more thumb wheels. Alternatively, a cross-section of shaft 502 may be illustrated by FIG. 4. Joystick 512 may be intuitively steered, particularly if a camera catheter image is in a random orientation to the orientation of distal end 504 of instrument 500. Joystick 512 may be actuated by squeezing proximal plate 516 and distal plate 514 together in a direction of deflection opposite of the desired direction of deflection of distal end 504. For example, if proximal plate 516 is squeezed together with distal plate 514 at the upper portion of proximal plate 516 and distal plate 514, then distal end 504 and the forceps will be deflected downwards. Instrument 500 includes handle 518 including holes 520 through each of which a finger of one hand of an operator, for example the index and middle fingers, may be inserted to grasp handle 518. Lever 522 includes a hole 524 through which another finger of the hand of the operator, for example a thumb, may be inserted, to translate lever 522 distally inward into handle 518 to open forceps or to translate lever 522 proximally outward from handle 518 to close forceps.

Referring to FIG. 6, a perspective view of another example of a joystick 600 is illustrated. Joystick 600 is distal to handle 604 and proximal to flexible shaft 602. Joystick 600 includes a bellows 612 extending between distal plate 614 and proximal plate 616.

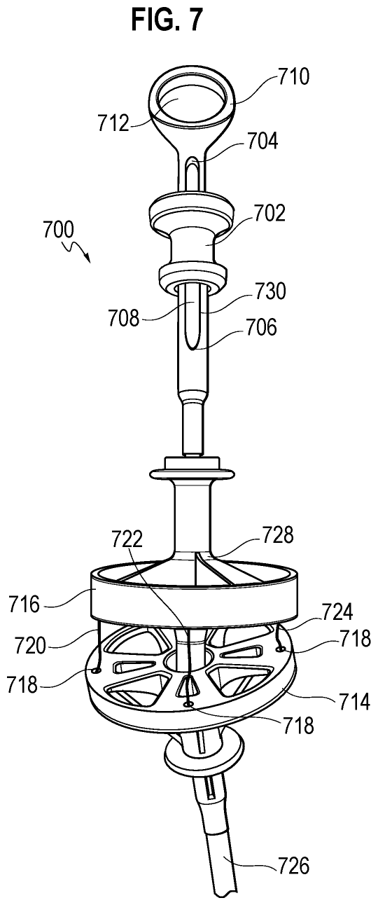

Referring to FIG. 7, a partial perspective view of another example of a steerable endoscopic instrument 700 is illustrated. Shaft 726 may be made of a flexible material and have a distal end connected to a steering mechanism. Examples of steering mechanisms may include joystick 728 as illustrated in FIG. 7, a rack that may operate with a pinion, and one or more thumb wheels. Alternatively, a cross-section of shaft 726 may be illustrated by FIG. 4. Joystick 728 may be intuitively steered, particularly if a camera catheter image is in a random orientation to the orientation of the distal end of instrument 700. Joystick 728 includes proximal plate 716 and distal plate 714. Distal plate 714 includes four deflection bores 718 (three bores 718 are shown in FIG. 7) through distal plate 714 from the proximal surface to the distal surface of distal plate 714, near the circumference of distal plate 714 and evenly spaced about the circumference of distal plate 714. Each of four steering wires 720, 722, and 724 (fourth steering wire is not shown in FIG. 7) extend from the distal surface of proximal plate 716 through one of the deflection bores 718 and may connect to the distal end of shaft 726. Joystick 728 may be actuated by squeezing proximal plate 716 and distal plate 714 together in a direction of deflection opposite of the desired direction of deflection of the distal end. For example, if proximal plate 716 is squeezed together with distal plate 714 near steering wire 720, then the distal end of shaft 726 will be deflected in the direction of steering wire 724 due to proximal force on steering wire 724 and on the distal end of shaft 726. Instrument 700 includes handle 710 including hole 712, through which a finger of one hand of an operator, for example the thumb, may be inserted. Actuator 702 may be translated distally and proximally along slot 730, which has distal proximal end 704 and distal end 706. An operator may grasp actuator 702 between two fingers, such as between the middle and index fingers, in order to move actuator 702 along slot 730. Attached to a distal end of actuator 702 is a proximal end of drive wire 708. A distal end of drive wire 708 may be attached to a driver, which may be configured to control aspects of instrument 700. For example, distal translation of actuator 702 results in distal translation of drive wire 708. The distal end of drive wire 708 and the driver may cause, for example, forcep jaws to open by a mechanism such as a lever system, or by a pinion gear rotating over teeth of a rack. Proximal translation of actuator 702 results in proximal translation of drive wire 708 and the driver, which may cause, for example, forcep jaws to close.

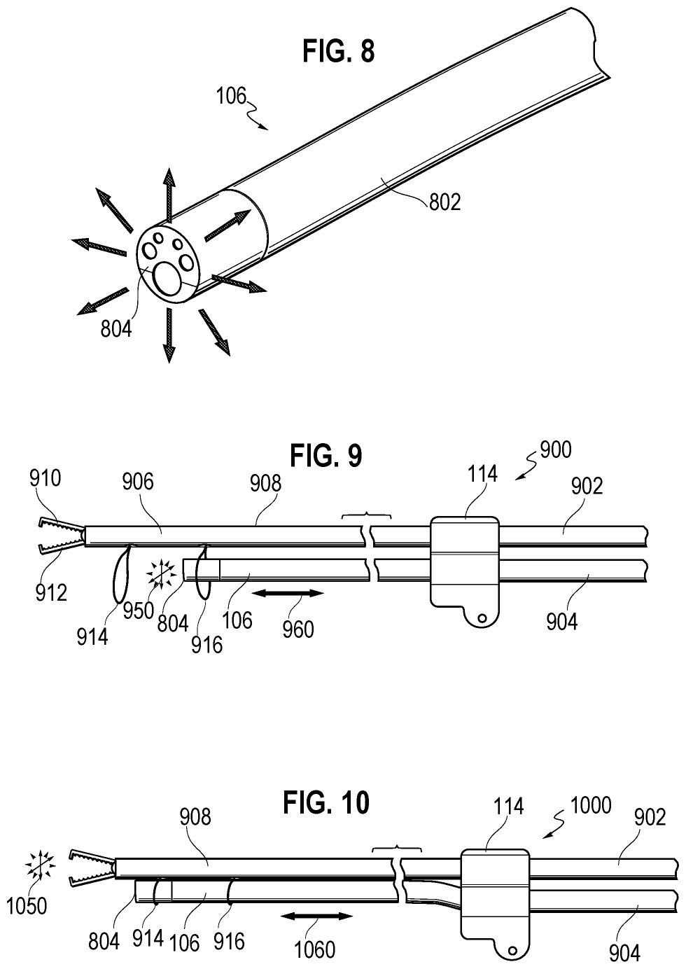

Referring to FIG. 8, a partial perspective view of a distal instrument end 802 of an example of a camera catheter 106 is illustrated. Camera catheter 106 has distal surface 804 that may be steered by being deflected in various directions relative to a longitudinal proximal-distal axis of camera catheter 106, including more than two, three, four, six, eight, twelve, twenty-four, or thirty-six directions, and up to 360 directions relative to a longitudinal proximal-distal axis of camera catheter 106. The degree of deflection relative to a longitudinal proximal-distal axis of camera catheter 106 may be less than 180 degrees, or less than 175 degrees, or less than 170 degrees, or less than 165 degrees, or less than 160 degrees, or less than 155 degrees, or less than 150 degrees, or less than 145 degrees, or less than 140 degrees, or less than 135 degrees, or less than 130 degrees, or less than 125 degrees, or less than 120 degrees, or less than 115 degrees, or less than 110 degrees, or less than 105 degrees, or less than 100 degrees, or less than 95 degrees, or less than 90 degrees. Camera catheter 106 may be wire-guided. Alternatively, camera catheter 106 may have a cross-section as illustrated in FIG. 4, whereby two or more steering wires extend through the length of steering lumens within camera catheter 106. Camera catheter 106 steering may be used to direct and position a side-by-side instrument at a desired location. A coupling mechanism is provided in an instrument, to couple distal end 802 of camera catheter 106 to a distal end of a side-by-side instrument, such as forceps, for use in the biliary tree. The instrument to be steered could be a wire-guided type device.

Referring to FIG. 9, a partial side view of yet another example of a distal portion 900 of an endoscope system. Camera catheter 106 may be translated proximally and distally relative to pivot arm 114 and second accessory channel 904 according to bidirectional arrow 960. Distal surface 804 may be deflected in various directions relative to a proximal-distal longitudinal axis parallel to bidirectional arrow 960, as illustrated by arrow 950, including more than two, three, four, six, eight, twelve, twenty-four, or thirty-six directions, and up to 360 directions relative to a proximal-distal longitudinal axis parallel to bidirectional arrow 960. The degree of deflection relative to a proximal-distal longitudinal axis parallel to bidirectional arrow 960 may be less than 180 degrees, or less than 175 degrees, or less than 170 degrees, or less than 165 degrees, or less than 160 degrees, or less than 155 degrees, or less than 150 degrees, or less than 145 degrees, or less than 140 degrees, or less than 135 degrees, or less than 130 degrees, or less than 125 degrees, or less than 120 degrees, or less than 115 degrees, or less than 110 degrees, or less than 105 degrees, or less than 100 degrees, or less than 95 degrees, or less than 90 degrees. Shaft 906 of instrument 908 may be flexible and extends from a distal end of first accessory channel 902 and includes a coupling mechanism. Examples of coupling mechanisms may include one or more loops. The example of coupling mechanisms illustrated in FIG. 9 include first loop 914 and second loop 916. Camera catheter 106 may be translated such that distal surface 804 may pass through second loop 916 and first loop 914. Instrument 908 may be forceps including upper jaw 910 and second jaw 912.

Referring to FIG. 10, a partial side view of yet another example of a distal portion 1000 of an endoscope system is illustrated, wherein after distal surface 804 passes through second loop 916 and first loop 914, first loop 914 and second loop 916 may be pulled tight around camera catheter 106 to provide distal portion 1000. Instrument 908 and camera catheter 106 may thereafter be translated simultaneously relative to pivot arm 114, first accessory channel 902, and second accessory channel 904, according to bidirectional arrow 1060. Further, instrument 908 and camera catheter 106 may be deflected in various directions together, relative to a proximal-distal longitudinal axis parallel to bidirectional arrow 1060, to provide side-by-side steering of instrument 908 and camera catheter 106. In distal portion 1000, arrow 1050 indicates various directions of deflection for side-by-side steering of instrument 908 and camera catheter 106, including more than two, three, four, six, eight, twelve, twenty-four, or thirty-six directions, and up to 360 directions relative to a proximal-distal longitudinal axis parallel to bidirectional arrow 1060. The degree of deflection relative to a proximal-distal longitudinal axis parallel to bidirectional arrow 1060 may be less than 180 degrees, or less than 175 degrees, or less than 170 degrees, or less than 165 degrees, or less than 160 degrees, or less than 155 degrees, or less than 150 degrees, or less than 145 degrees, or less than 140 degrees, or less than 135 degrees, or less than 130 degrees, or less than 125 degrees, or less than 120 degrees, or less than 115 degrees, or less than 110 degrees, or less than 105 degrees, or less than 100 degrees, or less than 95 degrees, or less than 90 degrees.

Referring to FIG. 11, an example of an endoscope system 1100 is illustrated. Endoscope system 1100 may be generally shaped as an elongate tube including distal portion 100, central portion 1102, and proximal, or handle, portion 1104. Central portion 1102 may be a flexible, elongate tube 1106 with at least one lumen 1108 running throughout the length of central portion 1102. Central portion 1102 may connect distal portion 100 and proximal portion 1104 together. The at least one lumen 1108 of central portion 1102 may extend through distal portion 100 and handle portion 1104 of endoscope system 100 as well. Central portion 1102 may be made of a braided material, such as a polyester block amide (including, for example, PEBAX), with a polytetrafluoroethylene (“PTFE”) liner to provide sufficient torqueability and pushability. Other potential materials for central portion 1102 include, but are not limited to, polyethylene, polypropylene, and nylon.

The present disclosure also presents methods of performing endoscopy in a bile duct of a mammal. In an example, the present disclosure presents a method for performing endoscopy in a bile duct of a mammal, including: providing a scope system including an elongate tube defining a lumen extending longitudinally therethrough, the elongate tube including a distal portion; at least one accessory channel including at least one tubular structure defining an accessory lumen extending longitudinally therethrough, the at least one accessory channel movably disposed at least partially within the lumen of the elongate tube; a first steerable endoscopic instrument extending at least partially through the at least one accessory channel, the first steerable endoscopic instrument including a first distal instrument end that is movable relative to a distal channel end of the at least one accessory channel; and a second steerable endoscopic instrument extending at least partially through the at least one accessory channel, the second steerable endoscopic instrument including a second distal instrument end that is movable relative to the distal channel end of the at least one accessory channel; and steering the first distal instrument end and the second distal instrument end separately within the bile duct.

In another example of a method for performing endoscopy in a bile duct of a mammal, the second steerable endoscopic instrument may include a cholangioscope and the first steerable endoscopic instrument may be selected from the group consisting of a suction device, an irrigation device, an insufflation device, a camera lens washing device, an electrohydraulic lithotripsy probe, forceps, a basket, snares, electrosurgical devices, wires, a dilation balloon, an extraction balloon, a needle knife, hemostasis clips, and laser-based devices.

In yet another example of a method for performing endoscopy in a bile duct of a mammal, the method may further include: translating the second distal instrument end distally through a loop on the first distal instrument end; tightening the loop around the second distal instrument end; and steering the first distal instrument end and the second distal instrument end together within the bile duct.

Although the present disclosure has been described with reference to examples and the accompanying drawings, the present disclosure is not limited thereto, but may be variously modified and altered by those skilled in the art to which the present disclosure pertains without departing from the spirit and scope of the present disclosure.

The subject-matter of the present disclosure may also relate, among others, to the following aspects:

A first aspect relates to a scope system, comprising: an elongate tube defining a lumen extending longitudinally therethrough, the elongate tube comprising a distal portion; at least one accessory channel comprising at least one tubular structure defining an accessory lumen extending longitudinally therethrough, the at least one accessory channel movably disposed at least partially within the lumen of the elongate tube; a first steerable endoscopic instrument extending at least partially through the at least one accessory channel, the first steerable endoscopic instrument comprising a first distal instrument end that is movable relative to a distal channel end of the at least one accessory channel; and a second steerable endoscopic instrument extending at least partially through the at least one accessory channel, the second steerable endoscopic instrument comprising a second distal instrument end that is movable relative to the distal channel end.

A second aspect relates to the scope system of aspect 1, wherein the first steerable endoscopic instrument comprises a cholangioscope.

A third aspect relates to the scope system of any preceding aspect, wherein the first distal instrument end is configured to deflect from a longitudinal axis of the elongate tube in at least two directions, wherein a deflection in each of the at least two directions is less than an angle of 180 degrees relative to the longitudinal axis.

A fourth aspect relates to the scope system of any preceding aspect is selected from the group consisting of a suction device, an irrigation device, an insufflation device, a camera lens washing device, an electrohydraulic lithotripsy probe, forceps, a basket, snares, electrosurgical devices, wires, a dilation balloon, an extraction balloon, a needle knife, hemostasis clips, and laser-based devices.

A fifth aspect relates to the scope system of any preceding aspect, wherein the second steerable endoscopic instrument comprises a steerable sheath.

A sixth aspect relates to the scope system of any preceding aspect, wherein the second steerable endoscopic instrument comprises an outer sheath comprising two or more steering wires.

A seventh aspect relates to the scope system of any preceding aspect, wherein the second distal instrument end is configured to deflect from a longitudinal axis of the elongate tube in at least two directions, wherein a deflection in each of the at least two directions is less than an angle of 180 degrees relative to the longitudinal axis.

An eighth aspect relates to the scope system of any preceding aspect, wherein the first steerable endoscopic instrument and/or the second steerable endoscopic instrument comprises a steering mechanism.

A ninth aspect relates to the scope system of aspect 8, wherein the steering mechanism is selected from the group consisting of a joystick, a rack and pinion, and a thumb wheel.

A tenth aspect relates to the scope system of any preceding aspect, wherein the second steerable endoscopic instrument comprises forceps.

An eleventh aspect relates to the scope system of aspect 10, wherein the second steerable endoscopic instrument comprises an actuator or a lever configured to open and close a first jaw and a second jaw of the forceps.

A twelfth aspect relates to the scope system of any preceding aspect, wherein the scope system comprises: at least two accessory channels, each of the at least two accessory channels comprising at least one tubular structure defining an accessory lumen extending longitudinally therethrough, the at least two accessory channels movably disposed at least partially within the lumen of the elongate tube; and a coupling mechanism configured to couple the first distal instrument end to the second distal instrument end; and wherein the first steerable endoscopic instrument extends at least partially through a first accessory channel of the at least two accessory channels, the first steerable endoscopic instrument comprising the first distal instrument end that is movable relative to the distal channel end of the at least two accessory channels; and wherein the second steerable endoscopic instrument extends at least partially through a second accessory channel of the at least to accessory channels, the second steerable endoscopic instrument comprising the second distal instrument end that is movable relative to the distal channel end.

A thirteenth aspect relates to the scope system of aspect 12, wherein the coupling mechanism comprises a loop on the first distal instrument end, the loop configured to envelop and tighten around the second distal instrument end or a loop on the second distal instrument end that is configured to envelop and tighten around the first distal instrument end.

A fourteenth aspect relates to a method for performing endoscopy in a bile duct of a mammal, comprising: providing a scope system comprising: an elongate tube defining a lumen extending longitudinally therethrough, the elongate tube comprising a distal portion; at least one accessory channel comprising at least one tubular structure defining an accessory lumen extending longitudinally therethrough, the at least one accessory channel movably disposed at least partially within the lumen of the elongate tube; a first steerable endoscopic instrument extending at least partially through the at least one accessory channel, the first steerable endoscopic instrument comprising a first distal instrument end that is movable relative to a distal channel end of the at least one accessory channel; and a second steerable endoscopic instrument extending at least partially through the at least one accessory channel, the second steerable endoscopic instrument comprising a second distal instrument end that is movable relative to the distal channel end of the at least one accessory channel; and steering the first distal instrument end and the second distal instrument end separately within the bile duct.

A fifteenth aspect relates to the method of aspect 14, wherein the second steerable endoscopic instrument comprises a cholangioscope; and wherein the first steerable endoscopic instrument is selected from the group consisting of a suction device, an irrigation device, an insufflation device, a camera lens washing device, an electrohydraulic lithotripsy probe, forceps, a basket, snares, electrosurgical devices, wires, a dilation balloon, an extraction balloon, a needle knife, hemostasis clips, and laser-based devices.

A sixteenth aspect relates to the method of aspect 13 or 14, further comprising: translating the second distal instrument end distally through a loop on the first distal instrument end; tightening the loop around the second distal instrument end; and steering the first distal instrument end and the second distal instrument end together within the bile duct.

In addition to the features mentioned in each of the independent aspects enumerated above, some examples may show, alone or in combination, the optional features mentioned in the dependent aspects and/or as disclosed in the description above and shown in the figures.

What is claimed is:

1. A scope system, comprising:
an elongate tube defining a lumen extending longitudinally therethrough, the elongate tube comprising a distal portion;
at least one accessory channel comprising at least one tubular structure defining an accessory lumen extending longitudinally therethrough, the at least one accessory channel movably disposed at least partially within the lumen of the elongate tube;
a first steerable endoscopic instrument extending at least partially through the at least one accessory channel, the first steerable endoscopic instrument comprising a first distal instrument end that is movable relative to a distal channel end of the at least one accessory channel; and
a second steerable endoscopic instrument extending at least partially through the at least one accessory channel, the second steerable endoscopic instrument comprising a second distal instrument end that is movable relative to the distal channel end.

2. The scope system of claim 1, wherein the first steerable endoscopic instrument comprises a cholangioscope.

3. The scope system of claim 1, wherein the first distal instrument end is configured to deflect from a longitudinal axis of the elongate tube in at least two directions, wherein a deflection in each of the at least two directions is less than an angle of 180 degrees relative to the longitudinal axis.

4. The scope system of claim 1, wherein the second steerable endoscopic instrument is selected from the group consisting of a suction device, an irrigation device, an insufflation device, a camera lens washing device, an electrohydraulic lithotripsy probe, forceps, a basket, snares, electrosurgical devices, wires, a dilation balloon, an extraction balloon, a needle knife, hemostasis clips, and laser-based devices.

5. The scope system of claim 1, wherein the second steerable endoscopic instrument comprises a steerable sheath.

6. The scope system of claim 1, wherein the second steerable endoscopic instrument comprises an outer sheath comprising two or more steering wires.

7. The scope system of claim 1, wherein the second distal instrument end is configured to deflect from a longitudinal axis of the elongate tube in at least two directions, wherein a deflection in each of the at least two directions is less than an angle of 180 degrees relative to the longitudinal axis.

8. The scope system of claim 1, wherein the first steerable endoscopic instrument and/or the second steerable endoscopic instrument comprises a steering mechanism.

9. The scope system of claim 8, wherein the steering mechanism is selected from the group consisting of a joystick, a rack and pinion, and a thumb wheel.

10. The scope system of claim 1, wherein the second steerable endoscopic instrument comprises forceps.

11. The scope system of claim 10, wherein the second steerable endoscopic instrument comprises an actuator or a lever configured to open and close a first jaw and a second jaw of the forceps.

12. A scope system, comprising:
an elongate tube defining a lumen extending longitudinally therethrough, the elongate tube comprising a distal portion;
at least two accessory channels, each of the at least two accessory channels comprising at least one tubular structure defining an accessory lumen extending longitudinally therethrough, the at least two accessory channels movably disposed at least partially within the lumen of the elongate tube;
a first steerable endoscopic instrument extending at least partially through a first accessory channel of the at least two accessory channels, the first steerable endoscopic instrument comprising a first distal instrument end that is movable relative to a distal channel end of the at least two accessory channels;
a second steerable endoscopic instrument extending at least partially through a second accessory channel, the second steerable endoscopic instrument comprising a second distal instrument end that is movable relative to the distal channel end; and
a coupling mechanism configured to couple the first distal instrument end to the second distal instrument end.

13. The scope system of claim 12, wherein the coupling mechanism comprises a loop on the first distal instrument end, the loop configured to envelop and tighten around the second distal instrument end, or a loop on the second distal instrument end that is configured to envelop and tighten around the first distal instrument end.

14. The scope system of claim 12, wherein the first steerable endoscopic instrument comprises a cholangioscope.

15. The scope system of claim 12, wherein the second steerable endoscopic instrument is selected from the group consisting of a suction device, an irrigation device, an insufflation device, a camera lens washing device, an electrohydraulic lithotripsy probe, forceps, a basket, snares, electrosurgical devices, wires, a dilation balloon, an extraction balloon, a needle knife, hemostasis clips, and laser-based devices.

16. The scope system of claim 12, wherein the first steerable endoscopic instrument and/or the second steerable endoscopic instrument comprises a steering mechanism selected from the group consisting of a joystick, a rack and pinion, and a thumb wheel.

17. The scope system of claim 12, wherein the second steerable endoscopic instrument comprises forceps and a lever configured to open and close a first jaw and a second jaw of the forceps.

18. A method for performing endoscopy in a bile duct of a mammal, comprising:
providing a scope system comprising:
an elongate tube defining a lumen extending longitudinally therethrough, the elongate tube comprising a distal portion;
at least one accessory channel comprising at least one tubular structure defining an accessory lumen extending longitudinally therethrough, the at least one accessory channel movably disposed at least partially within the lumen of the elongate tube;
a first steerable endoscopic instrument extending at least partially through the at least one accessory channel, the first steerable endoscopic instrument comprising a first distal instrument end that is movable relative to a distal channel end of the at least one accessory channel; and
a second steerable endoscopic instrument extending at least partially through the at least one accessory channel, the second steerable endoscopic instrument comprising a second distal instrument end that is movable relative to the distal channel end of the at least one accessory channel; and
steering the first distal instrument end and the second distal instrument end separately within the bile duct.

19. The method of claim 18, wherein the second steerable endoscopic instrument comprises a cholangioscope; and
wherein the first steerable endoscopic instrument is selected from the group consisting of a suction device, an irrigation device, an insufflation device, a camera lens washing device, an electrohydraulic lithotripsy probe, forceps, a basket, snares, electrosurgical devices, wires, a dilation balloon, an extraction balloon, a needle knife, hemostasis clips, and laser-based devices.

20. The method of claim 18, further comprising:
translating the second distal instrument end distally through a loop on the first distal instrument end;
tightening the loop around the second distal instrument end; and
steering the first distal instrument end and the second distal instrument end together within the bile duct.
